(19) United States
(12) Patent Application Publication
(10) Pub. No.: US 2006/0025859 A1
(43) Pub. Date: Feb. 2, 2006

(54) Breast prosthesis

(75) Inventors: Nils Stelter, Achenmuhle (DE); Helmut Wild, Roseheim (DE)

Correspondence Address: DILWORTH & BARRESE, LLP, 333 EARLE OVINGTON BLVD., UNIONDALE, NY, 11553, US

(73) Assignee: Amoena, Raubling (DE)

(21) Appl. No.: 11/044,697

(22) Filed: Jan. 27, 2005

(30) Foreign Application Priority Data
Jul. 30, 2004 (DE) UM 202004011988.9

(51) Int. Cl. A61F 2/52

(52) U.S. Cl. 623/7

(57) ABSTRACT

The invention relates to a breast prosthesis substantially consisting of bodies welded in plastic films and modeled on the shape of the breast, preferably of an addition cross-linked two-component silicone rubber mass, on whose rear side, in accordance with the invention, a separate and exchangeably insert can be put in or on.

The invention relates to a breast prosthesis substantially consisting of bodies welded into plastic films and modeled on the shape of the breast, preferably of an addition cross-linked two-component silicone rubber mass.

A method is known, for example from DE 27 01 627 A, of manufacturing breast prostheses from shell-shaped bodies welded into plastic films, modeled on the shape of the breast and from an addition cross-linked two-component silicone rubber mass. The prostheses manufactured with this method are almost ideal copies of the natural breast in their appearance and in their behavior due to the elastic softness, the movement, the consistence and the weight of the material used. The breast prostheses are attached to the breast of the wearer in a manner as non-slipping as possible. For this purpose, it is known, for example from EP 392 930 A, to provide breast prostheses of the type first named with a peripheral shoulder formed by a step on their rear side inside a peripheral lip-shaped rim, with adhesive strips or adhesive pieces being fastened to said peripheral shoulder which cooperate with the adhesive regions of strips fastened to the body of the woman by skin-friendly adhesive means such that the prosthesis is connected to the holding strip adhering to the body and can be released from it again. A hook and loop connection is, for example, provided as the fastening means.

It has become known, for example from DE 44 21 516 C1, for the improvement of the comfort in wear of the aforesaid breast prostheses to provide a breast prosthesis with a textile rear side in which a hollow space between the textile rear side and the prosthesis consisting of plastic can be filled with cotton wool.

A further development is known from EP 1,232,733 B. It is described here that a so-called PCM material is added to the silicone rubber mass of the breast prosthesis. Such a PCM material can, for example, consist of a paraffin substance having a melting range from approximately 33-37° C. The so-called heat paraffins used as the PCM material are paraffins especially modified for technical heat applications. These heat paraffins have a specific melting heat or melting enthalpy which is very high for organic substances at 130-150 kJ/kg. If the skin surface temperature increases beyond this temperature level, the PCM material is melted. A correspondingly high melting energy is required for the melting which is taken away from the skin surface and thereby contributes to a pleasant cooling effect.

Further modifications of generic breast prostheses have become know which partly consist of making the breast prosthesis as a multi-layer prosthesis, with the part of the breast prosthesis close to the body being filled with a thixotropic material.

The variety of the breast prostheses now known means that the orthopedic stores which sell these breast prostheses have to operate large and cost-intensive warehousing.

The consumer must furthermore always make a compromise in choosing the breast prosthesis. If, for example, she selects a prosthesis with a cooling material, this is advantageous in summer and during sports, but not on other occasions.

It is now the object of the invention to provide a breast prosthesis which is as multi-functional as possible and which meets different demands.

This object is solved in accordance with the invention by the combination of the features of claim 1. The core of the invention consists of the breast prosthesis now being made in a modular manner. This is achieved in that a separate and exchangeable insert can be placed into or onto the rear side of the breast prosthesis.

In accordance with the invention, the material can be selected in dependence on the area of application. Preferred aspects of the invention result from the subordinate claims dependent on the main claim.

A recess can advantageously be provided on the rear side into which the insert can be inserted.

The corresponding insert will advantageously consist of materials welded into plastic which take over the respective function to be met.

Accordingly, a PCM material can be chosen as the material of the insert which has a phase transition temperature in the range of body temperature. This insert can therefore bring about a corresponding cooling effect.

Alternatively, a thixotropic or thixotropicized material can be selected as the material of the insert which can adapt particularly advantageously to the scar region of the consumer.

A further alternative for the insert consists of it being filled with soft silicone.

The insert can advantageously also have a cavity and recesses which are adapted to a scar region.

For simple separation, the insert is connected to the breast prosthesis by releasable connection elements such as a hook and loop connection or a glue/adhesive connection.

The insert can have a plurality of welds on its surface, whereby the flexibility of the insert is increased.

An air-permeable textile layer can be provided on the rear side of the breast prosthesis with an inserted insert. It can likewise again be releasably placed one the rear side of the breast prosthesis and of the insert inserted therein. This textile layer, which consists of a so-called three-dimensional mesh permits circulation of air and therefore contributes to the comfort in wear.

In accordance with a further preferred aspect of the invention, the insert can have a self-adhesive layer on its rear side which permits a fastening of the total breast prosthesis.

The aforesaid air-permeable textile layer can also be arranged directly on the rear side of the insert, i.e. it can be fixedly connected to it.

The insert can have the usual substantially triangular shape. It can, however, also advantageously be designed substantially in the form of a symmetrical ellipse.

The breast prosthesis itself can be made as a single-layer prosthesis and advantageously also as a two-layer prosthesis, with all known materials usually used with breast prostheses being able to be used as the materials for the filling of the one layer or two layers.

Further details and advantages of the invention will be explained in more detail with reference to embodiments shown in the drawing. There are shown:

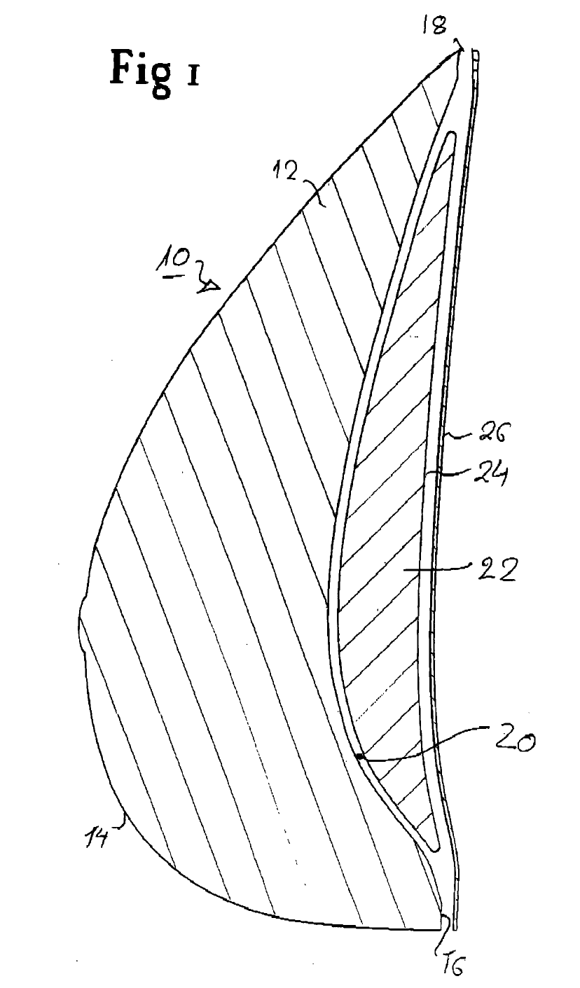

FIG. 1: the design of a breast prosthesis in accordance with a first embodiment variant of the invention;

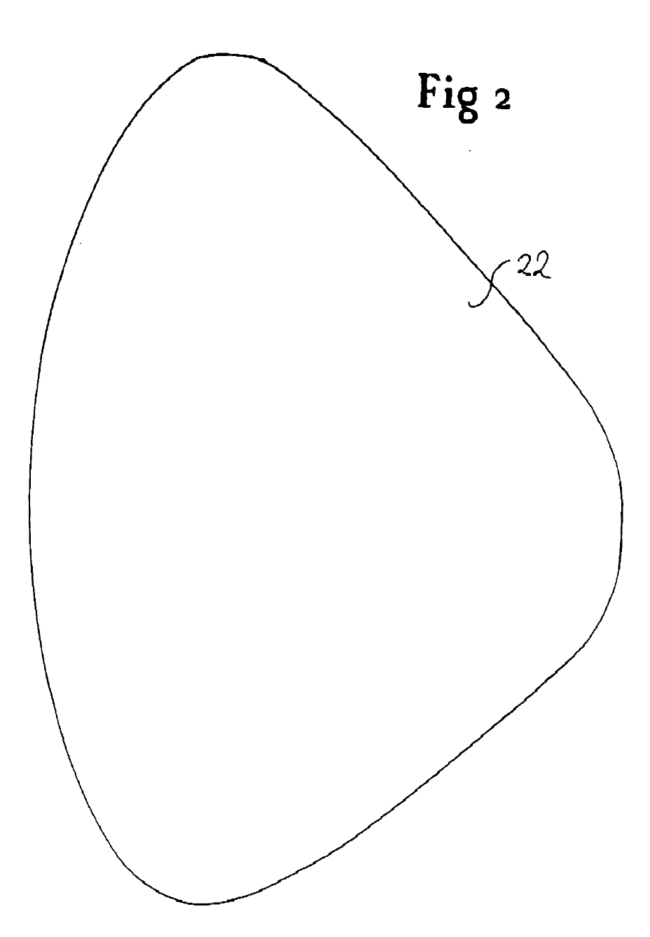

FIG. 2: the outline of an insert for the breast prosthesis in accordance with FIG. 1;

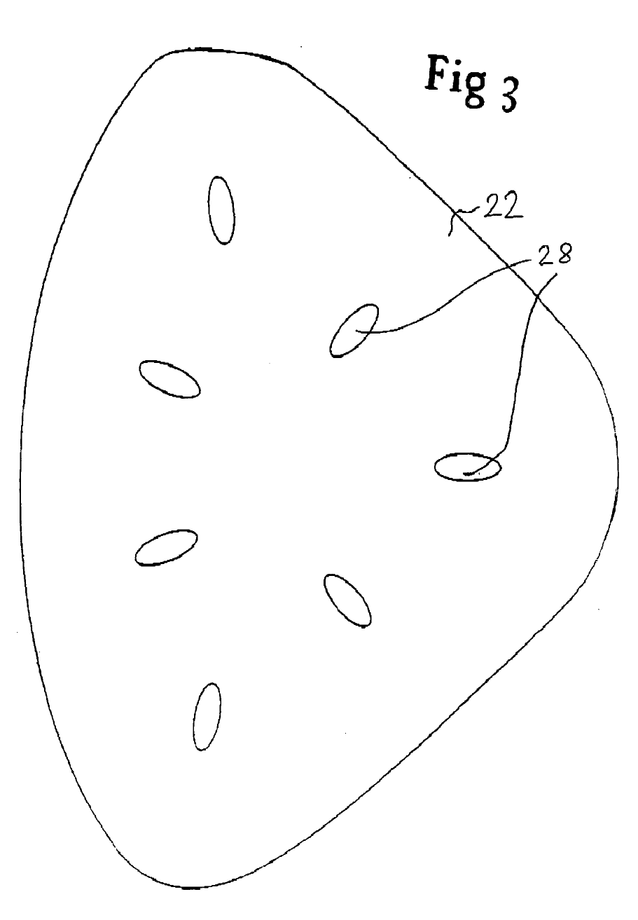

FIG. 3: an outline of an insert in accordance with FIG. 2 in a modified embodiment;

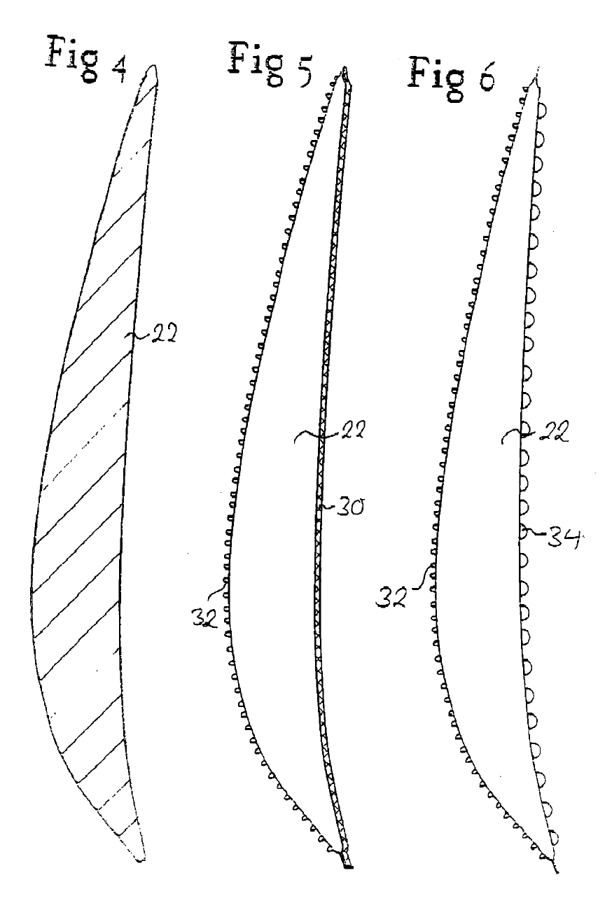

FIG. 4: a longitudinal section through an insert in accordance with FIG. 2;

FIG. 5: a modified form of the insert in accordance with FIG. 4;

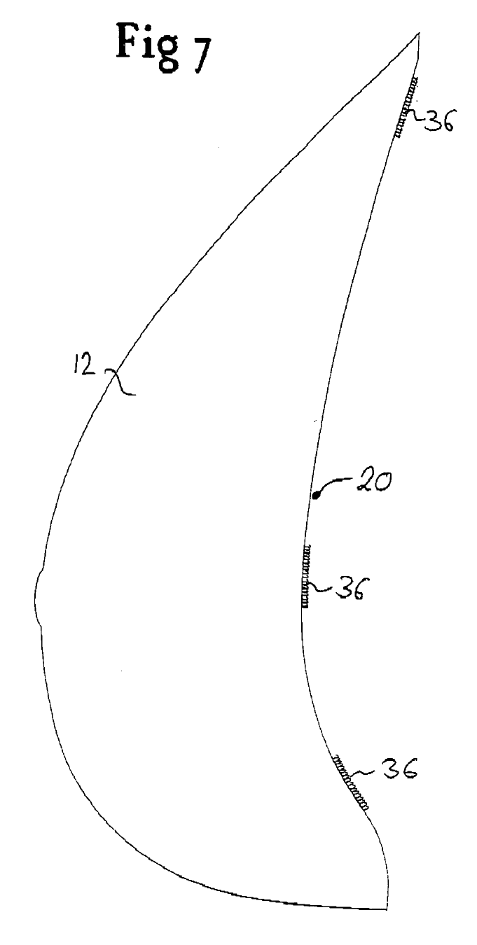

FIG. 6: a modified insert similar to the insert in accordance with FIG. 4;

FIG. 7: a breast prostheses with a corresponding recess for the insertion of an insert not shown here; and

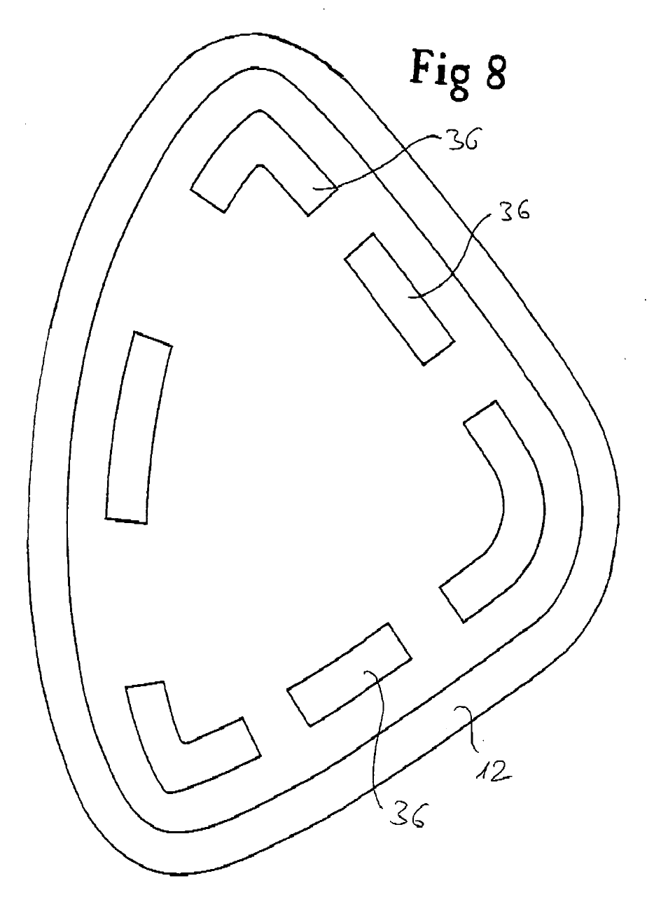

FIG. 8: the plan view of the breast prosthesis in accordance with FIG. 7 from the rear.

The breast prosthesis 10 shown in FIG. 1 consists of a shell-shaped body 12 made of a soft-elastically set addition cross-linked two-component rubber mass whose outside is covered by a polyurethane film 14 and whose inside is covered by a polyurethane film 16 which are connected to one another along a common peripheral rim 18 by a peripheral welding seam.

In the example shown here, a recess 20 is provided on the rear side of the breast prosthesis 10 into which a separate and exchangeable insert 22 can be inserted.

The insert 22 is likewise enveloped in a polyurethane film 24. It can consist of different materials.

It can thus, for example, consist of a PCM material which has a phase transition temperature in the region of body temperature. If it consists of such a material, a plurality of PCM inserts can be added as basic fittings to a prosthesis in order, for example, for the inserts to be used changeably on particularly hot days.

Alternatively, the insert can also consist of a thixotropic or thixotropicized material. An insert of soft silicone can also be selected.

In the embodiment in accordance with FIG. 1, a textile layer 26 is arranged on the rear side of the prosthesis and of the insert 22 received in the recess 20, said textile layer consisting of a so-called three-dimensional mesh, that is of a three-dimensional net structure, which is air-permeable and thus serves a pleasant comfort in wear. This textile layer 26 can be connected to the prosthesis 12 or to the insert 22 by an adhesive layer in a manner in which it can be released again.

FIG. 2 shows the outlines of an insert 22 for the embodiment of a symmetrical triangular prosthesis.

In FIG. 3, an insert 22 in accordance with FIG. 2 is shown on whose polyurethane envelope welds 28 are provided which increase the flexibility of the insert 22.

In FIG. 4 there is a longitudinal section through a typical insert 22 which shows that the insert is immediately brought in the shape adapted to the recess 20. Alternatively, the insert 22 could, however, also consist of a symmetrical elliptical shape. This symmetrical elliptical shape would also permit an insertion into the recess 20, since the material adapts to the pre-determined wall. The manufacturing effort would hereby be reduced.

In the alternative embodiment variant in accordance with FIG. 5, a textile layer 30 is fixedly applied directly to the rear side of the insert 22. The handling capability has hereby been further facilitated since here fewer parts have to be put together by the wearer before the breast prosthesis is put on. A type of fleece material 32 is laminated on the convex front side of the insert 22 and permits a connection to corresponding hook and loop parts on the rear side of the breast prosthesis 12 if these are provided with a hook and loop connection.

An adhesive layer 34, for example of adhesive silicone, is applied to the body side of the modified insert 22 shown here in FIG. 6 and the total breast prosthesis 10 can be adhesively attached to the skin of the wearer via this. A fleece material 32 is again also provided here on the opposite side.

FIG. 7 shows a breast prosthesis 12 with the corresponding recess 20 in which hook and loop parts 36 are provided which cooperates with the fleece material of an insert 22 such as is shown in FIGS. 5 and 6.

In FIG. 8, a plan view of the prosthesis in accordance with 12 is shown from behind in which the arrangement of the hook and loop connection strips can be seen in detail.

1. A breast prosthesis composed of bodies welded into plastic films and modeled on the shape of the breast, preferably of an additional cross-linked two-component natural rubber mass,
wherein
a separate and exchangeable insert can be placed in or on its rear side.

2. A breast prosthesis in accordance with claim 1, wherein it has a recess into which the exhangeable insert can be inserted.

3. A breast prosthesis in accordance with claim 1, wherein the insert is composed of materials welded into plastic films.

4. A breast prosthesis in accordance with claim 3, wherein PCM material is chosen as the material of the insert which has a phase transition temperature in the region of body temperature.

5. A breast prosthesis in accordance with claim 3, wherein a thixotropic or thixotropicized material is selected as the material of the insert.

6. A breast prosthesis in accordance with claim 3, wherein a soft silicone is selected as the material of the insert.

7. A breast prosthesis in according with claim 1, wherein the insert can have cavities and recesses.

8. A breast prosthesis in accordance with claim 1, wherein the insert is connected to the breast prosthesis via releasable connection elements such as a hook and loop connection or a glue/adhesive connection.

9. A breast prosthesis in accordance with claim 1, wherein the insert has a plurality of welds on its surface.

10. A breast prosthesis in accordance with claim 1, wherein the rear side of the breast prosthesis with an inserted insert is covered by an air-permeable textile layer, preferably by a three-dimensional mesh.

11. A breast prosthesis in accordance with claim 1, wherein the insert has a self-adhesive layer on its rear side.

12. A breast prosthesis in accordance with claim 1, wherein an air-permeable textile layer is arranged directly on the rear side of the insert.

13. A breast prosthesis in accordance with claim 1, wherein the insert is substantially designed in the shape of a symmetrical ellipse.

14. A breast prosthesis in accordance with claim 1, wherein it is made as a two-layer prosthesis.

15. A breast prosthesis in accordance with claim 2, wherein the insert is composed of materials welded into plastic films.

16. A breast prosthesis in accordance with claim 15, wherein PCM material is chosen as the material of the insert which has a phase transition temperature in the region of body temperature.

17. A breast prosthesis in accordance with claim 15, wherein a thixotropic or thixotropicized material is selected as the material of the insert.

18. A breast prosthesis in accordance with claim 15, wherein a soft silicone is selected as the material of the insert.

19. A breast prosthesis in accordance with claim 18, wherein the rear side of the breast prosthesis with an inserted insert is covered by an air-permeable textile layer, preferably by a three-dimensional mesh.

20. A breast prosthesis in accordance with claim 17, wherein the rear side of the breast prosthesis with an inserted insert is covered by an air-permeable textile layer, preferably by a three-dimensional mesh.
